(19) United States
(12) Patent Application Publication
(10) Pub. No.: US 2025/0032050 A1
(43) Pub. Date: Jan. 30, 2025

(54) Smart ICM ECG Filtering

(71) Applicant: BIOTRONIK SE & Co. KG, Berlin (DE)

(72) Inventors: Antje LINNEMANN, Berlin (DE); Bjoern Henrik DIEM, Berlin (DE); Benjamin JAIDI, Garching (DE); Ronny AMBERG, Berlin (DE); Ravi Kiran Kondama REDDY, Portland, OR (US); R. Hollis WHITTINGTON, Portland, OR (US)

(73) Assignee: BIOTRONIK SE & Co. KG, Berlin (DE)

(21) Appl. No.: 18/715,213

(22) PCT Filed: Dec. 9, 2022
(86) PCT No.: PCT/EP2022/085239

§ 371 Date: May 31, 2024

Related U.S. Application Data
(60) Provisional application No. 63/288,720, filed on Dec. 13, 2021.

(30) Foreign Application Priority Data
Jan. 11, 2022 (EP) 22150910.2

(51) Int. Cl. A61B 5/00; A61B 5/0245; A61B 5/353; A61B 5/361; G06N 20/00

(52) U.S. Cl. CPC A61B 5/7221; A61B 5/0245; A61B 5/353; A61B 5/361; A61B 5/686; G06N 20/00

(57) ABSTRACT

A method for analyzing an event determined by an implantable device. The method comprises: verifying the event at least in part based on a heart episode associated with the event; wherein the verifying is based at least in part on a type of the event.

### CROSS-REFERENCE TO RELATED APPLICATIONS

This application is the United States National Phase under 35 U.S.C. § 371 of PCT International Patent Application No. PCT/EP2022/085239, filed on Dec. 9, 2022, which claims the benefit of European Patent Application No. 22150910.2, filed on Jan. 11, 2022 and U.S. Provisional Patent Application No. 63/288,720, filed on Dec. 13, 2021, the disclosures of which are hereby incorporated by reference herein in their entireties.

### TECHNICAL FIELD

The present invention generally relates to a method for analyzing an event determined by an implantable device, a corresponding apparatus, a corresponding system and a corresponding computer program.

### BACKGROUND

Various methods for determining an event associated with a heart activity are currently known in the field. Such methods may be implemented by an implantable device that is implanted into a patient to monitor the patient's heart. For example, the implantable device may comprise an implantable cardiac monitor (ICM), a pacemaker, a cardioverter defibrillator, etc. Usually, the implantable device may be configured to sense/determine a heart activity (e.g., an electrocardiogram, ECG, signal) and based thereon determine if a particular event (e.g., a specific heart arrythmia) is present. For example, an according medical response may be triggered (e.g., alerting medical personnel, alerting the patient to undergo a detailed medical check-up, an immediate medical intervention in case of an imminent cardiac arrest, etc.).

However, in the currently known methods incorrectly determined events (e.g., false positives) may not be excluded. This may result in an inadequate medical response since medical personnel is incorrectly informed, which may lead to wrong medical treatment of the patient. Moreover, this may also result in unnecessary patient unease since the patient can be wrongfully alarmed of a (not actually present) harmful cardiac event.

Hence, the currently known techniques do not always lead to an optimal analyzing of events determined by an implantable device. Therefore, there is a need to find ways to improve analyzing of determined events.

The aspects describe herein address the above need at least in part.

The present disclosure is directed toward overcoming one or more of the above-mentioned problems, though not necessarily limited to embodiments that do.

### SUMMARY

A first aspect relates to a method for analyzing an event determined by an implantable device. The method comprises verifying the event at least in part based on a heart episode associated with the event, wherein the verifying is based at least in part on a type of the event.

Hence, the event determined by the implantable device is additionally verified. The (additional) verification may thus serve as a double check if the event was correctly determined. Moreover, the (additional) verification of the event may depend on the type of the determined event. The inventors have found out that this can greatly improve the (additional) verification result compared to exclusively verifying every event merely by the same verification steps. The present invention may thus enable a verifying based on the special characteristics of the type of the event in contrast to other generic verification steps (e.g., other generic verifications may only consider a certain parameter but not the type of the event). To illustrate an example of this concept, if a determined event is of type A, the event is verified via a verification A. If a determined event is of a different type B, the event is verified via a different verification B.

The event may comprise that a statistical anomaly has occurred in the heart rate of the patient. For example, the event may comprise a Boolean associated with the event (e.g., a positive/a negative, true/false, etc.) that indicates whether or not an event was determined. For example, the event may be related to a certain heart arrythmia (e.g., an atrial arrhythmia, a ventricular arrhythmia, a junctional arrhythmia, etc.). To that regard, the type of the event may comprise a specific type of the certain heart arrythmia (e.g., a specific type of atrial arrhythmia, a specific type of ventricular arrhythmia, a specific type of junctional arrhythmia, etc.).

The heart episode associated with the event may be the heart activity over a specific time duration. The heart episode may also be considered a time window of a heart activity in which the event occurs. For example, the heart activity may comprise an electrocardiogram, ECG, signal (and/or any cardiac vector signal). To that regard, the heart episode may comprise an electrocardiogram, ECG, signal (and/or any cardiac vector signal) over a specific time duration wherein the event occurs in said specific time duration. The heart episode may, for example, be defined by the time duration between a start time t₀ and an ending time t_{E}. The specific time duration of the heart episode may be a clinically relevant time duration needed to determine the event (and/or the type of the event). In another example the specific time duration of the heart episode may be chosen/adapted for technical purposes to ease determining of the event (e.g., the time duration may be fixed at 40 to 80 seconds, e.g., 50 to 70 seconds, or 60 seconds, 1 hour, etc. wherein the event is determined in the fixed time duration). In other words, the heart episode may be the heart activity (e.g., an ECG signal) over a certain time period in which the event occurs or in which the event is analyzed. In an example the method may also comprise sensing the heart activity by the implantable device (e.g., via an implantable sensor).

In an example the type comprises at least one of the following: an atrial fibrillation onset, an asystole, a high ventricular rate, a bradycardia. Atrial fibrillation onset may be considered a new or first detectable onset of atrial arrhythmia. Asystole may be considered an absence of ventricular contractions and/or a prolonged pause between ventricular contractions. The high ventricular rate (HVR) may be considered an unusually high ventricular heart rate from a medical perspective. Bradycardia may be considered an unusually low heart rate from a medical perspective. In some examples the type comprises at least one element of the group consisting of the following elements: an atrial fibrillation onset, an asystole, a high ventricular rate, a bradycardia.

In an example the verifying comprises determining whether the event is true positive and/or whether the event is false positive. For example, the (initial) determination of the event by the implantable device may be considered as a positive. The (additional) verifying may comprise determining whether the event is indeed a true positive or actually a false positive. If the event is verified as a true positive the event can be considered to have been correctly determined by the implantable device. However, if the verification result of the verifying is a false positive the event can be considered to have been incorrectly determined by the implantable device.

In some examples the verifying is performed by a second device, e.g., a device different from the implantable device. The verifying may thus not necessarily be performed by the implantable device that has determined the event. This may enable an improved verifying since limitations of the implantable device can be overcome by the second device. For example, an implantable device is usually limited to a certain (e.g., narrow) size to ensure optimal medical compatibility once implanted into patient. However, this size constraint can lead to hardware and power supply restrictions of the implant which may only allow limited resources regarding computational power. These limitations can be overcome by performing the verifying via the second device which, for example, may not be an implantable device. The second device may thus be configured to comprise more computational power than the implantable device. In another example the second device may not be limited by the power supply restrictions (e.g., a battery lifetime) that are associated with an implantable device residing in a patient. In an example the second device may be an external computing device (e.g., a smartphone, a desktop pc, a server (system), a cloud, etc.).

In an example the method comprises receiving information associated with the event from the implantable device. For example, the information may be received by the second device (as outlined herein). The method may further comprise sending information associated with the event to the second device, e.g., from the implantable device (e.g., wirelessly and/or directly or indirectly). The information associated with the event may, for example, comprise the heart episode (e.g., the ECG signal as outlined herein), the type of the event, the Boolean associated with the event, a notifier that an event was determined, data associated with the implantable device (e.g., an identification/serial number and/or a model type of the implantable device, patient information (e.g., a patient identification number, a patient name, etc.)).

In another example the verifying comprises upsampling of the heart episode (e.g., an upsampling of the ECG signal). The upsampling may comprise increasing the sample rate of the heart episode by a factor of at least two, at least five, at least eight, for example, eight or ten (e.g., the upsampling may comprise increasing the sampling rate from 128 Hz to 1024 Hz). For example, the heart episode may be received from the implantable device at a relatively low sampling, and then be upsampled to a relatively high sampling rate (e.g., by the second device). The upsampling may also comprise applying various interpolation steps (e.g., an interpolation filter). In another example the verifying comprises an anti-aliasing (of the signal) of the heart episode.

In an example the verifying comprises detecting a QRS information (e.g., a QRS interval and/or a QRS complex) of the heart episode. To that regard, initially QRS blocks (i.e., QRS candidates) may be detected as regions of interests wherein a QRS block may be a time window of the heart episode that substantially comprises a QRS information. For example, the QRS information may be detected according to the Elgendi algorithm. Subsequently, for each of the QRS blocks the QRS peak (e.g., a R wave peak) can be determined according to the present invention. The QRS peak may be determined based on applying a global filter function onto the QRS block to create a squared bandpass signal (which can also be an output of the Elgendi algorithm). Details regarding the Elgendi algorithm may be found in “Elgendi M. (2013). Fast ORS detection with an optimized knowledge-based method: evaluation on 11 standard ECG databases. PloS one. 8(9). e73557”. However, in contrast to the known Elgendi algorithm, the present invention may comprise that the QRS peak is determined based on finding a (local) peak (i.e., a (local) maximum) of the squared bandpass signal (or a (local) maximum of a slope (of the squared bandpass signal)) and which is positioned furthest to the left inside the QRS block (wherein left may refer to an earlier time of the QRS block compared to another time in the QRS block). The present invention may further comprise that the QRS peak is subsequently associated with said determined (local) peak of the squared bandpass signal. Accordingly, a time position in the QRS block and/or a time position in the heart episode can be accorded to the determined QRS peak. In other words, the inventive method may implement that a QRS peak is associated with a local peak in the QRS complex wherein the local peak has the highest local peak height (e.g., a highest value difference from the local peak maximum to an adjacent local minimum) and/or has the highest (absolute) peak amplitude and/or wherein the local peak is preferred which is the furthest to the left of the QRS complex. In contrast, in the Elgendi algorithm an R Peak is detected based on a brute force search that considers a frequency band, event related durations, and an offset fraction. This in turn, leads to a time-consuming algorithm that requires a database used for training and optimization. The inventive method may thus overcome such disadvantages by considering the local peaks of the squared bandpass filer, their respective slopes and preferring the left most local peak position inside a QRS complex (or QRS block) to determine the QRS peak.

In an example of the method if the type comprises an atrial fibrillation onset, the verifying comprises detecting. in the heart episode, a (potential) P wave peak by determining a sliding linear regression in a vicinity of a (predetermined) QRS peak. The vicinity may be a time region preceding the QRS peak and may be determined based on a time interval between the QRS peak and its preceding QRS peak. The QRS peak (and/or its preceding QRS peak) may be determined as stated herein or may be determined by any other suitable method known in the field. For example, detecting the (potential) P wave peak may also be based on an advanced QRS complex removal. To illustrate an example, initially the vicinity of the QRS peak may be determined based on the detected QRS blocks and determined QRS peaks (as outlined herein). The QRS peak may serve as a reference point (i.e., a point of origin) when applying signal processing onto the heart episode signal in the vicinity of the QRS peak. The vicinity may also be referred to as the area of interest for the P wave peak. A slope correction may be implemented in the vicinity via the sliding linear regression, which in turn removes global slopes and (indirectly) enhances local slopes. The remaining (local) peaks can be subsequently sorted based on their features to find a (potential) P wave peak. For example, the P wave peak may be associated with a peak having the highest local peak height (e.g., a highest value difference from the peak maximum to an adjacent local minimum) and/or having the highest (absolute) peak amplitude, and/or being furthest away from the reference point (i.e., the point of origin, i.e., the corresponding QRS peak).

In an example of the method the verifying (of the atrial fibrillation onset event) is further based at least in part on one of the following: an RR interval, a PP interval, an RP interval, a correlation of a QRS complex morphology and/or on an ectopy probability, in the heart episode. For example, the verifying may also comprise determining at least one of the following: the RR interval, the PP interval, the RP interval, in the heart episode by a suitable method known in the field. For example, the RR interval (e.g., the RR intervals of the heart episode) may be calculated based on the determined QRS peaks (e.g., by calculating the time difference between subsequent QRS peaks). The verifying may subsequently comprise extracting statistical features that are based on the outlined heart intervals (e.g., the RR, PP, RP interval). The statistical features may relate to statistical characteristics of the heart intervals (e.g., mean values, maximum/minimum values, etc.). The verifying may also comprise determining a correlation of the QRS complex morphology and/or the ectopy probability (i.e., ectopic beat probability). To illustrate an example the correlation of the QRS complex morphology may comprise an autocorrelation of at least one QRS complex morphology, and/or a cross-correlation between QRS complex morphologies of different QRS complexes. For example, the cross-correlation may be performed for morphologies of QRS complexes of the same heart episode, and/or for a morphology of a QRS complex of the heart episode relative to a morphology of a reference QRS complex. The determining of the ectopy probability may be based on irregularities in heart intervals (as outlined herein) compared to one or more features of other heart intervals in the heart episode (e.g., the ectopy probability may be based on (irregularities in) the RR intervals in the heart episode: e.g., irregularities may be a deviation by a predetermined threshold from an average value, for example). When the verifying is performed by the second device the ectopy probability may be determined according to an algorithm which is also used to determine the ectopy probability on the implantable device. However, in that case, the ectopy probability used for verifying may be based on QRS peaks detected by the second device (e.g., as outlined herein).

In an example of the method the verifying is performed by an artificial intelligence system and/or machine learning system that has been trained with events that were verified via manual inspection of heart episodes. The training may be implemented via historical data with known labels (e.g., true positive, false positive). In an example, the artificial intelligence system and/or the machine learning system may be inputted with one or more statistical features of the outlined heart intervals as outlined herein, the correlation of the QRS complex as outlined herein and/or the ectopy probability as outlined herein to perform the verifying based thereon. The machine learning model may comprise, for example, XGBoost.

In an example of the method, if the type comprises an asystole, a high ventricular rate and/or a bradycardia, the verifying may comprise eliminating, from the heart episode, RR intervals associated with a clipping of an amplitude (or associated with signal saturation), and preferably also RR intervals adjacent to at least one RR interval associated with a clipping of an amplitude. In particular, RR intervals that start with amplitude clipping must be eliminated. RR intervals that only end with amplitude clipping do not have to be eliminated and can be considered further.

The clipping of the amplitude may be defined by the absolute value of the amplitude of the heart episode (e.g., the ECG signal) exceeding a threshold value. The threshold value may, for example, be associated with an unusually large value which does not have a medical reason (e.g., the clipping of the amplitude may be caused by technical reasons, e.g., sensing errors, etc.). In another example, the clipping may be defined in that the amplitude of the heart episode exceeds a certain percentage of the threshold value over a clipping period. For example, the percentage may be X % (e.g., 65%), the threshold value may be C_{TH} (e.g., 2 V) wherein the clipping period may be t_{clip} (e.g., 100 ms), so that if the amplitude exceeds X % of the threshold value C_{TH} for a clipping period of t_{clip} a clipping of the amplitude is determined. The RR interval associated with the clipping (i.e., a clipped amplitude) may then be eliminated as outlined herein, and preferably also RR intervals adjacent to that associated with the clipping.

In an example the verifying (of the asystole event) further comprises: detecting, in the heart episode, whether a pause associated with the asystole is present between subsequent QRS peaks, preferably corresponds to a RR interval that has not been eliminated (as outlined herein). The pause may be considered an unusually prolonged pause (e.g., longer than 3s) between subsequent QRS peaks. The pause may be determined based on QRS peaks detected via the method of the first aspect as outlined herein.

In a further example the verifying (of the asystole event) further comprises: detecting, in the heart episode, whether a pause associated with the asystole is present between subsequent RR intervals, preferably between subsequent RR intervals that have not been eliminated (as outlined herein). The pause may be considered an unusually prolonged pause between subsequent RR intervals. The pause may be determined based on QRS peaks detected via the method of the first aspect as outlined herein.

To illustrate another example, the implantable device may generally be configured to determine the pause according to the asystole algorithm which may be based on QRS peaks which were detected by the implantable device via an internal QRS method (e.g., a suitable method known in the field). For example, the pause may be determined by the implantable device for determining the asystole (or another event). When in turn the verifying of the asystole is performed (as outlined herein) the pause may be determined via the same asystole algorithm (as was implemented by the implantable device). However, in that case, the present invention may comprise that the determination of the pause is based on QRS peaks detected via the method of the first aspect (as outlined herein) and not via the internal QRS method (implemented by the implantable device). Since the calculation capabilities of the implantable device may be limited the internal QRS method may be also limited in algorithmic complexity which could result in less accurate QRS peak detection and therefore in wrongful determination of the pause (and thus the asystole). The outlined exemplary method thus ensures that the pause is determined based on a highly accurate QRS peak detection which enables that the verifying is based on a more accurate QRS peak detection. This may ensure that asystole events can be reliably verified and overcoming the limits of the implantable device.

In an example the verifying (of the asystole event) further comprises: if a pause is detected determining that the event is true positive otherwise the event is determined as false positive.

The inventive method may thus enable that by eliminating the clipped RR intervals the determined pause represents an actually present pause. The determined pause can thus not be associated with a technical malfunction of the implantable device, for example, but an actual event. In turn, if no pause is determined by the method of the first aspect this may indicate that the implantable device has incorrectly determined an event.

In a further example, the verifying may not necessarily depend on eliminating RR intervals. In that case, the method may comprise detecting, in the heart episode, whether a pause associated with the asystole is present between subsequent RR intervals or QRS peaks and if the pause is not present determining that the event is false positive. Hence, this may indicate without eliminating RR intervals that the implantable device has incorrectly detected pauses associated with asystole. This may enable determining a false positive event without necessarily determining a clipping of an amplitude in the heart episode (as outlined herein).

In an example of the method, if the type comprises a high ventricular rate and/or a bradycardia, the verifying comprises a calculation of a heart rate based on the heart episode. The heart rate may comprise the number of contractions of the heart per minute. The heart rate may be calculated based on determining the number of QRS peaks for a certain time period wherein the result may be further processed to gain the number of QRS peaks per minute (and thus the heart rate per minute). In an example, the heart rate may be calculated based on counting the number of QRS peaks of the entire heart episode. It is also conceivable that the heart rate is calculated over a certain number of heart beats or over a certain heart rate time window that is smaller than the heart episode duration.

To illustrate another example of the method, the implantable device may generally be configured to calculate the heart rate according to an internal algorithm which may be based on QRS peaks which were detected by the implantable device via an internal QRS method (e.g., a suitable method known in the field). For example, the heart rate may be determined by the implantable device for determining an event. When in turn the verifying of the event is performed (as outlined herein) the heart rate may be determined via the same internal algorithm. However, in that case, the present invention may comprise that the calculation of the heart rate is based on QRS peaks detected via the method of the first aspect (as outlined herein) and not via the internal QRS method. Since the calculation capabilities of the implantable device may be limited the internal QRS method may be limited in algorithmic complexity as well which could result in less accurate QRS peak detection and therefore in wrongful determination of the heart rate and an associated event. The outlined exemplary method thus ensures that the heart rate is determined based on a highly accurate QRS peak detection which enables that the verifying is based on a more accurate QRS peak detection.

In an example, the verifying is based at least in part on comparing the calculated heart rate with a predetermined threshold. For example, if the type comprises high ventricular heart rate and the calculated heart rate is above the predetermined threshold the event may be determined as a true positive (and otherwise as a false positive). For example, if the type comprises bradycardia and the calculated heart rate is below the predetermined threshold the event may be determined as a true positive (and otherwise as a false positive). The predetermined threshold may be different for the verifying of high ventricular heart rate and the verifying of bradycardia. For example, a first predetermined threshold for verifying high ventricular heart rate may be 100 beats per minute (bpm) wherein the predetermined threshold for verifying bradycardia may be 60 bpm.

In another example, the verifying is based at least in part on the (calculated) heart rate exceeding (or being below) the predetermined threshold for a specific duration. For example, if the type comprises high ventricular heart rate and the heart rate is above the predetermined threshold (e.g., 100 bpm) for the specific duration (e.g., 9 s), the event may be determined as true positive. For example, if the type comprises bradycardia and the heart rate is below the predetermined threshold (e.g., 60 bpm) for the specific duration (e.g., 10 s), the event may be determined as true positive.

In another example, the verifying is based at least in part on the (calculated) heart rate exceeding (or being below) the predetermined threshold for a specific number of heart beats. For example, if the type comprises high ventricular heart rate and the heart rate is above the predetermined threshold (e.g., 100 bpm) for the specific number of heart beats (e.g., 15 heart beats), the event may be determined as true positive. For example, if the type comprises bradycardia and the heart rate is below the predetermined threshold (e.g., 60 bpm) for the specific number of heart beats (e.g., 10 heart beats), the event may be determined as true positive.

Considering the specific duration and/or the specific number of heart beats may ensure that the verifying is more accurate. For example, this enables that a (medically) unusual heart rate over a relatively short period of time and/or over a relatively low number of heart beats can be screened out since it may not be medically relevant.

A second aspect relates to an apparatus, e.g., computer or computer system, for analyzing an event determined by an implantable device, wherein the apparatus is configured to perform a method according to the first aspect. In an example the apparatus (e.g., computer) may be comprised in the second device. For example, the apparatus may comprise a smartphone, a desktop pc, a server (system), a cloud, etc. Another aspect relates to an implantable device for determining an event wherein the implantable device is configured to perform the method of the first aspect (as outlined herein). The implantable device may be further configured to transmit information associated with the event to the computer of the second aspect.

A third aspect relates to a system for analyzing an event determined by an implantable device. The system comprises an apparatus (as outlined herein) and the implantable device.

A fourth aspect relates to a computer program comprising instructions to perform a method according to the first aspect (as outlined herein), when the instructions are executed.

It is noted that the method steps as described herein may include all aspects described herein, even if not expressly described as method steps but rather with reference to an apparatus (or device or system or computer). Moreover, the devices and computer programs as outlined herein may include means for implementing all aspects as outlined herein, even if these may rather be described in the context of method steps.

Whether described as method steps, computer program and/or means, the functions described herein may be implemented in hardware, software, firmware, and/or combinations thereof. If implemented in software/firmware, the functions may be stored on or transmitted as one or more instructions or code on a computer-readable medium. Computer-readable media include both computer storage media and communication media including any medium that facilitates transfer of a computer program from one place to another. A storage medium may be any available media that can be accessed by a general purpose or special purpose computer. By way of example, and not limitation, such computer-readable storage media can comprise RAM, ROM, EEPROM, FPGA, CD/DVD or other optical disk storage, magnetic disk storage or other magnetic storage devices, or any other medium that can be used to carry or store desired program code means in the form of instructions or data structures and that can be accessed by a general-purpose or special-purpose computer, or a general-purpose or special-purpose processor.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

FIG. 1 Schematic representation of an exemplary embodiment of a system comprising an implantable device and a computer system according to the present invention.

FIG. 2 Schematic representation of an exemplary embodiment of a method according to the present invention.

### DETAILED DESCRIPTION

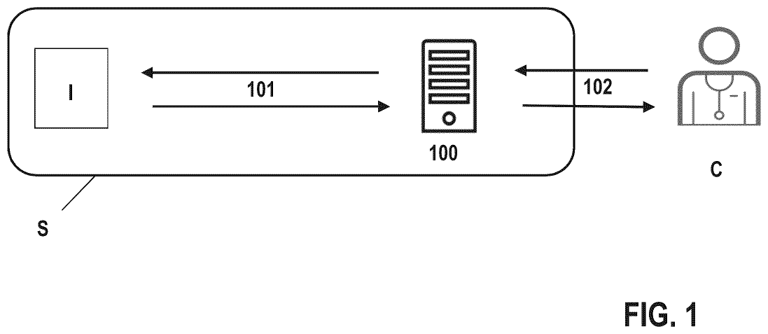

FIG. 1 shows a schematic representation of an exemplary embodiment of a system S comprising an implantable device I and an apparatus, such as a computer (system) 100, according to the present invention. It is noted that an implantable device as referred to herein may relate to a device that can be implanted but has not yet been implanted into a patient but also to a device that has already been implanted into a patient.

For example, the implantable device I may comprise an implantable cardiac monitor, ICM, an implantable electrophysiology device, as well as any other implant capable of sensing a heart activity (e.g., a pacemaker, a cardioverter defibrillator, an implantable sensor, etc.). For example, if the implantable device I is an ICM may be implantable under the skin of a patient. The implantable device I may be configured to monitor and/or record heart activity of a patient's heart. The heart activity may comprise an electrocardiogram, ECG, signal, or any other cardiac vector signal.

The system S may further comprise a computer (system) 100. The computer (system) 100 may comprise a remote service center which comprises various computational nodes (e.g., computers, servers, databases, etc.). In an example the computer (system) 100 may be part of the remote service center. The remote service center may enable a remote access and data management of the information/data that has been acquired by the implantable device I. The computer (system) 100 may also comprise peripheral interfaces to various devices other than the implantable device I to communicate with the remote service center. The implantable device I and the computer (system) 100 may be communicatively coupled via a communication link 101. The communication link 101 may enable a direct communication of information from the implantable device I to the computer (system) 100, and vice versa.

It is also conceivable that the communication link 101 may comprise an indirect communication path over one or more communication nodes (e.g., devices). For example, the implantable device 100 may communicate (e.g., transmit) information to the computer (system) 100 via an intermediary device. The computer (system) 100 may thus receive information from the implantable device I via the intermediary device. In accordance, the computer (system) 100 may communicate (e.g., transmit) information to the implantable device I via the intermediary device. The intermediary device may thus function as an intermediary communication node to enable the communication link 101. The intermediary device may comprise a programmer device, a cardiac messenger device, a smartphone, etc. For example, the programmer device may be used to adapt settings of the implantable cardiac monitor (and function as an intermediary node). The cardiac messenger device may be used to function as the main intermediary node to enable a regular communication of the patient's heart monitoring data from the implantable device I to the computer (system) 100. The cardiac messenger device may thus be a portable device that the patient carries with him/her.

The computer (system) 100 may be communicatively coupled via a communication link 102 for external readout by, for example, medical personnel C. The communication link 102 may be bidirectional and implemented between the computer (system) 100 and a communication node (not shown) that may be accessed by the medical personnel C. The medical personnel C may thus access heart activity data (e.g., monitoring data) of the patient if the according data has been communicated from the implantable device I to the computer (system) 100. The medical personnel may, for example, access the patient's heart activity (e.g., the ECG signal), as well as an event associated with a heart episode of the patient, the heart episode itself and/or the type of the event (as outlined herein). The computer (system) 100 may also receive information form the medical personnel C via the communication link 102. This may enable an import of clinical data associated with the patient and/or a setting of parameters used by computer (system) 100, as described herein.

The computer (system) 100 may be further communicatively coupled with various peripheral nodes that are not shown in FIG. 1. For example, the peripheral nodes may comprise a smartphone, that may be coupled to various other sensor (that may indicate medical sensory data of the patient). The peripheral node mode may also comprise an IT support node (for technical adjustments of the computer (system) 100), a physician's web portal, etc.

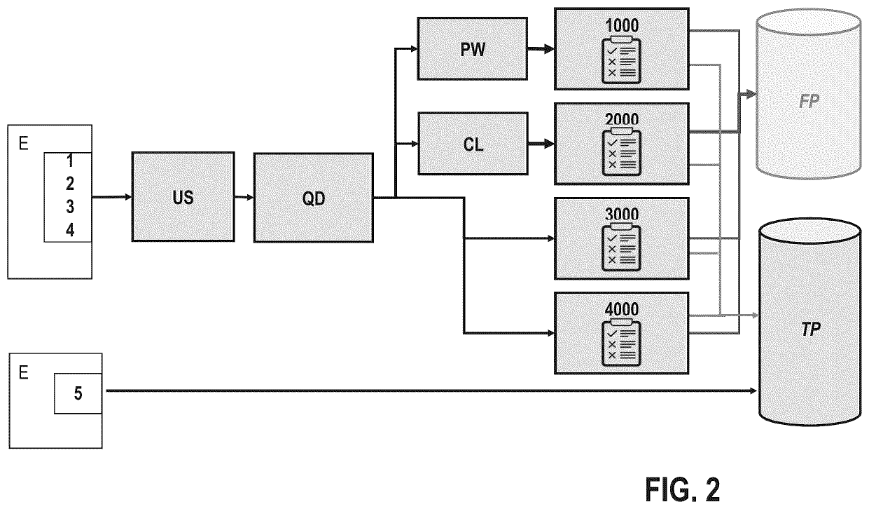

FIG. 2 shows a schematic representation of an exemplary embodiment of a method according to the present invention. The method may be implemented by computer (system) 100 (of FIG. 1). Initially, the implantable device I may be configured to determine an event E associated with a heart episode of the patient. The heart episode (as outlined herein) may be the heart activity (e.g., the ECG signal) over a specific time duration wherein the event takes place in the heart episode. The event E may comprise that an unusual or suspicious heart activity has occurred in the heart episode. For example, the event E may comprise a Boolean that indicates whether or not an unusual (or suspicious) heart activity has occurred in the heart episode. The presence of the event E (or the according Boolean) may function as flag that a certain heart episode is medically relevant. The implantable device I may further be configured to determine a type 1,2,3,4 of the event E. The type of the event may be a certain type of heart arrythmia. For example, the implantable device may be configured to determine that the type of the event E is an atrial fibrillation onset 1. The implantable device may also be configured to determine that type of the event E is an asystole 2. The implantable device may also be configured to determine that the type of the event E is a high ventricular rate 3. For example, the implantable device may also be configured to determine that the type of the event E is a bradycardia 4.

Accordingly, the event E and the type 1,2,3,4 of the event E are the starting point for the method 200 according to the present invention. The method 200 may ensure verifying the determined event E and/or the type of the event E. If the method is implemented by the computer (system) 100, the method 200 may initially comprise receiving information associated with the event E from the implantable device I (e.g., via the communication link 101 as outlined for FIG. 1). For example, the received information may comprise the event, type of the event, and the according heart episode (e.g., the according ECG signal, as outlined herein).

In other words, the method 200 may be used to verify a snapshot of an ECG signal (i.e., a certain time window of an ECG signal, i.e., the heart episode) for which the implantable device I has determined a particular type of event (e.g., an atrial fibrillation onset, an asystole, a high ventricular rate and/or a bradycardia). Subsequently, the heart episode will be mostly referred to as the ECG signal of the event E for exemplary purposes to illustrate the signal processing. The verification implemented by method 200 may thus determine the event E is false positive FP or true positive TP.

However, if the type of the event E is one of the types 1,2,3, or 4 signal processing and/or filtering is applied to the ECG signal of the event E, and in particular specific to the type of the event, e.g., it may be processed and/or filtered differently, depending on which type the event represents. The verifying is thus based on the type of the event E. Initially, a preprocessing is applied to every ECG signal of the event E of the type 1, 2, 3, 4. The preprocessing (not shown in FIG. 2) may comprise a single-phase edge smoothing, which can smooth plateaus, steps (e.g., stepped plateaus), single (point) peaks and/or valleys of the

ECG signal of the event E. The preprocessing may also comprise adapting the bit depth (i.e., sampling depth) of the ECG signal of the event E.

Subsequently an upsampling US may be performed. The upsampling US may comprise an interpolation (e.g., from 128 Hz to 1024 Hz). The upsampling US may adapt the ECG signal of the event E regarding bit depth and sampling frequency. The interpolation may occur via a combination of a sliding linear regression and a (subsequent) sliding average. The interpolated (i.e., upsampled) ECG signal of the event E may also be referred to as the reconstructed ECG signal of the event E. Furthermore, an error correction may be applied.

The error correction may repeat the downsampling implemented by the original device that acquired the original ECG signal of the event E (e.g., the implantable device I) on the reconstructed ECG signal of the event E to correct the reconstructed ECG signal of the event E. The error correction may thus minimize the deviations between the original ECG signal and the downsampled (original) signal.

Subsequently, a QRS peak detection QD is applied to every ECG signal of the event E of the type 1, 2, 3, 4. The QRS peak detection QD may be implemented as outlined herein. Initially, a QRS complex detection is implemented via a modified Elgendi-algorithm. The Elgendi-algorithm may detect regions of interests (i.e., so called QRS blocks of QRS candidates), wherein the QRS peaks are searched inside said regions. For the search a global filter function is applied to a QRS block. The filter function is configured to try to find the maximum in a squared bandpass signal associated with a QRS block of the ECG signal of the event E. The squared bandpass signal may be an output of the Elgendi-algorithm. In particular, the filter function is configured to find the (local) maximum, which has the highest slope and is positioned the furthest to the left inside the region of interest. Left, to that regard, may refer to an earlier time inside the region of interest. The goal is to find the (local) maximum (i.e., a local peak) with the highest peak height and the highest absolute signal value, wherein (local) maxima are preferred by the filter function that are positioned left in the region of interest. The most preferred peak according to the filter function may be determined as the QRS peak of the ECG signal of the event E. The method may further comprise that every QRS peak of the heart episode (e.g., the ECG signal of the event E) is detected as outlined herein. In other words the QRS peak detection QD may be referred to as being based on maximization of the squared bandpass filtered signal after modification with a slope aware left inclined Picard kernel.

Subsequently, the signal processing for verifying the event E is outlined for the type 1, 2, 3, or 4.

If the event E is of type 1 (i.e., an atrial fibrillation onset) a P wave detection PW is applied to the ECG signal of the event E to find a P wave candidate (which may be associated with the QRS block). To that regard, the region of interest is expanded to search for a P wave candidate. This may be done to find the P wave peak of the heart that usually precedes the QRS complex in time. According to the method, a time segment (e.g., RR interval) may be the time window between two subsequent QRS peaks. The two subsequent QRS peaks may be referred to according to their order in time as the first QRS peak and the second QRS peak. A percentage of the time segment constitutes a time region where the P wave candidate can be assumed. Said time region thus constitutes a region of interest (ROI) for finding the P wave candidate. Said time region may also be referred to herein as a vicinity of the QRS peak. The percentage may be variable according to the method (e.g., the percentage may be at least 10%, 20%, 40%, 50%, etc., e.g., the inventors have found out that about 30% to 50%, e.g., 40% achieved good results to find a P wave candidate). To illustrate an example, if the percentage is 50%, then only the second half of the time segment is associated with the second QRS peak and is thus the region of interest for finding the P wave candidate. The second half of the time segment is in this example thus a time region that directly precedes the second QRS peak. When searching for the P wave peak, the second QRS peak may serve as the point of origin (or as a reference coordinate). The P wave peak is thus searched in a negative time section since the region of interest for finding the P wave peak precedes the second QRS peak (i.e., the reference coordinate). To search for the P wave peak a slope correction is applied to the ECG signal of the event E in the region of interest for finding the P wave peak candidate. This slope correction may be based on a sliding linear regression. The sliding linear regression may comprise calculating a slope and one or more regression coefficients. By applying the slope correction global slopes are removed and local slopes are indirectly enhanced. The resulting peaks after the slope correction may enable that the P wave peak can be found more reliably. The resulting peaks may be sorted according to their features to determine the P wave peak. For example, the P wave peak may be determined to be the peak which has the highest peak height, the highest signal value and is furthest away from the second QRS peak (i.e., the point of origin, i.e., the reference coordinate). A potential P wave peak may also be the closest outwards extrema, from the slope with the greatest inclination (in relation to its length). In an example, an immediate time area preceding the QRS peak may not be considered in the region of interest for finding the P wave peak (e.g., the immediate vicinity may be associated with the Q wave of the heart).

Subsequently, the determined QRS peaks and the determined P wave peaks may be used to determine a statistical feature associated with the ECG signal of the event E. The QRS peaks and the P wave peaks may be used to determine heart intervals (e.g., RR intervals, PP intervals, RP intervals, etc.) as well as other features. The determined features may be used for verifying 1000 the event of type 1 (an atrial fibrillation onset), e.g., whether or not it is a true positive TP or a false positive FP. In the following some features are explained in more detail.

A feature may comprise an RR interval. The RR interval (or simply RR) may be a ID vector of length N−1 wherein N equals the number of R waves (or R wave peaks) (e.g., in the ECG signal of the event E). The RR interval may be defined by RRₙ=Rₙ₊₁−Rₙ (wherein n may numerically index R waves over the heart episode in their timely order, e.g., n may indicate an R wave and n+1 indicates the subsequent R wave).

Another feature may comprise RR_dist. RR_dist may consider the variance and correlation of quadratic means of differences between RR intervals. RR_dist may be a 2D matrix of size (N−2)×(N−2) wherein N equals the number of R waves (e.g., in the ECG signals of the event E). RR_dist may be calculated as follows: RR_dist=Cor(Sqrt((RRₘ−RRₘ₊₁)²+(RRₙ−RRₙ₊₁)²)).

A further feature may comprise dRR. dRR may consider the differentiation of RR intervals.

dRR may be a 1 D vector of length N−2, wherein N equals the number of R waves (e.g., in the ECG signal of the event E). dRR may be calculated as follows: dRRₙ=RRₙ₊₁−RRₙ ((wherein n may numerically index RR intervals over the heart episode in their timely order).

A further feature may comprise dRR_dist. dRR_dist may be similar as RR_dist but may consider dRR instead of RR. dRR_dist may be calculated as follows: dRR_distₘₙ=Cov(Sqrt((dRRₘ−dRRₘ₊₁)2+(dRRₙ−dRRₙ₊₁)2)).

A further feature may comprise RR_Rm. RR_Rm may consider a ratio of two neighboring intervals. RR RRm may be a 1D vector of length N−2 (N=number of R waves). RR_RRmn may be calculated as follows: RR_RRmₙ=RRₙ₊₁/RRₙ. (wherein n may numerically index RR intervals over the heart episode in their timely order).

A further feature may comprise RR_RRm_dist. RR_RRm_dist may calculated as RR_dist (as outlined herein) but with RR_RRm instead of RR.

A further feature may comprise an PP interval. The PP interval (or simply PP) may be a ID vector of length N−1 wherein N equals the number of P waves (or P wave peaks) (e.g., in the ECG signal of the event E). The PP interval may be defined by PPₙ=Pₙ₊₁−Pₙ (wherein n may numerically index P waves over the heart episode in their timely order, e.g., n may indicate a P wave and n+1 indicates the subsequent P wave).

A further feature may comprise a PR interval (or an RP interval). The PR interval may be the interval of the detected P wave peak (or the determined P wave candidate) to the closest subsequent R wave peak (e.g., QRS peak).

A further feature may comprise PQ_ratio. PQ_ratio may be a ratio of a number of found P wave candidates to a number of found R waves (e.g., number of found QRS peaks).

The outlined features associated with the heart episode may be statistically analyzed. The according statistics may result in respective statistical features. The statistical features may comprise at least one of the following: a median, a mean, a standard deviation, a ratio of standard deviation to mean, a quantile (e.g., 25% quantile, 50% quantile, 75% quantile), a range (e.g., the difference between a maximum and a minimum), a kurtosis. The statistical features may also be based on a histogram with fixed bin width. For example, the statistical features may comprise a max_bin (e.g., entries in max bin over all entries (=ratio in max) (fixed bin width)), numbins (e.g., number of bins !=0 (fixed bin width)), frac0bins (e.g., number of bins=0 over all bins (fixed bin width)), n2diff (e.g., average square difference from max bin count to the neighboring bin counts), etc.

A further feature may be RRautoCovRation, which may be a ratio of the largest to second largest autocovariance value of RR.

A further feature may be RRIntdInt_NEC and/or RRIntdInt_NEC Frac which is described in “Jie Lian. Lian Wang. Dirk Muessig. A Simple Method to Detect Atrial Fibrillation Using RR Intervals. The American Journal of Cardiology. Volume 107. Issue 10. 2011, Pages 1494-1497”.

A further feature may be RRDFA which may be based on a detrended fluctuation analysis (DFA) of RR.

A further feature may be QRS_tmpl_mean_maj which may be a mean correlation among QRS of a major group (from wave onset to wave offset).

A further feature may be QRS_tmpl_mean_min which may be a mean correlation among QRS of a minor group (from wave onset to wave offset).

A further feature may be Pcand_mean which may be a mean correlation among P wave candidates (from wave onset to wave offset)

A further feature may be ectopy_frac. The feature ectopy_frac may be a ratio of a number of ectopies to a number of regular QRS (intervals) (and may thus be considered an ecopy probability). An Ectopy may be counted if a first RR interval is less than or equal to a short threshold and a subsequent second interval is greater than or equal to a long threshold. Long and short thresholds may be updated for each QRS that was not counted as ectopy.

In summary, regarding the verifying of the event of type 1, the features herein outlined, as well as the statistical features (that may be based on the herein outlined features) may be a basis for verifying 1000 of the ECG signal of the event of type 1. As outlined herein these features may be inputted into a machine learning model and/or an artificial intelligence system. Subsequently the machine learning model (and/or the artificial intelligence system) may implement a binary classification of the event of type 1 into true positive TP or false positive FP. In an example the training data of the machine learning model is based on historical data wherein ECG signals of an event E of type 1 were classified into false positives and true positives by medical personnel (e.g., cardiac experts) via visual inspection. This may thus ensure an accurate verification of an event E of type 1 since medical expertise is considered.

As is shown in FIG. 2, if the event E is of type 2 (i.e., an asystole) a clipping detection CL is performed on the ECG signal of the event E after the QRS peak detection QD was performed. In some embodiments, also if the event E is of type 3 or 4 (i.e., high ventricular rate or bradycardia), the clipping detection CL may be performed on the ECG signal of the event E after the QRS peak detection QD was performed (not shown). In an example, after the clipping detection CL the verifying of the event of type 2 may comprise eliminating, from the ECG signal of the event E, RR intervals associated with a clipping of an amplitude (as outlined herein). The clipping detection CL may also be referred to as determining regions with prolonged positive or negative signal saturation. In an example also RR intervals adjacent to an RR interval that comprises a clipping of an amplitude are eliminated. In another example the RR intervals associated with a clipping (and preferably the adjacent RR intervals) may not be eliminated as such from the ECG signal of the event E but may be selected and/or marked. Hence, the “clipped” RR intervals may still be analyzed, but in view of the information that a clipping of the amplitude is present.

The verifying 2000 of the event E of type 2 may be based on detecting, in the ECG signal of the event E of type 2, whether a pause associated with the type 2 (i.e., the asystole) is present between subsequent QRS peaks, preferably corresponds to a RR interval that have not been eliminated or selected/marked (as outlined herein). The pause may be considered an unusually prolonged pause between subsequent QRS peaks (prolonged RR interval). The pause may be determined based on QRS peaks detected via the method as outlined herein.

However, the detection of the prolonged pause may also consider various other aspects (e.g., the features and/or the corresponding statistical features as outlined herein). For example, heart episodes with a detected pause may be considered as true positive TP and those without a detected pause may be false positive FP. In other examples, heart episodes with clipping within a detected pause can be classified as false positive FP, heart episodes without clipping within the pause can be classified as true positive TP. It may also be conceivable that episodes without any pause may be classified as false positive FP without the need to detect a clipping. In summary, the verifying 2000 of the event E of type 2 (as outlined herein) can determine whether the according ECG signal of the event E is false positive FP or true positive TP.

As is shown in FIG. 2, if the event E is of type 3 (i.e., high ventricular rate), after the QRS peak detection QD is performed the verifying 3000 of the according event E is executed as outlined herein. The verifying 3000 may comprise a calculation of a heart rate based on the ECG signal of the event E of type 3. Furthermore, the verifying 3000 may comprise comparing the calculated heart rate to a predetermined threshold (as described herein). This may enable to determine whether the event E of type 3 is false positive FP or true positive FP. For example, if the calculated heart rate exceeds a device programmed threshold (or a standardized threshold), the Event E of type 3 will be classified as true positive TP and otherwise as false positive FP.

As is shown in FIG. 2, if the event E is of type 4 (i.e., bradycardia), after the QRS peak detection QD is performed the verifying 4000 of the according event E is executed as outlined herein. The verifying 4000 may comprise a calculation of a heart rate based on the

ECG signal of the event E of type 4. Furthermore, the verifying 4000 may comprise comparing the calculated heart rate to a predetermined threshold (as described herein). This may enable to determine whether the event E of type 4 is false positive FP or true positive FP. For example, if the calculated heart rate falls below a device programmed threshold (or a standardized threshold), the event E of type 4 will be classified as true positive TP and otherwise as false positive FP.

For example, the implantable device may also be configured to determine further or less types of events.

For example, in the example of FIG. 2, the implantable device may also be configured to determine that the type of the event E is a type 5 meaning that the event is not of type 1, 2, 3 or 4 (i.e., meaning that the event is not an atrial fibrillation onset, not an asystole, not a high ventricular rate, not a bradycardia). Events of type 5, in the depicted example, will not be processed and are automatically treated as a relevant episode. Events of type 5 are thereby determined to be true positives TP. According to the method 200, if the type of the event is a type 5 the event E is determined to be true positive TP with no further signal processing and/or filtering. In other examples, also type 5 events and/or events of other types may be processed.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

1. Method for analyzing an event determined by an implantable device, the method comprising:
verifying the event at least in part based on a heart episode associated with the event;
wherein the verifying is based at least in part on a type of the event.

2. Method according to claim 1, wherein the type comprises at least one of the following: an atrial fibrillation onset, an asystole, a high ventricular rate, a bradycardia.

3. Method according to claims 1, wherein the verifying comprises determining whether the event is true positive and/or whether the event is false positive.

4. Method according to claim 1, further comprising receiving information associated with the event from the implantable device.

5. Method according to claim 1, wherein, if the type comprises an atrial fibrillation onset, the verifying comprises detecting, in the heart episode, a P wave peak by determining a sliding linear regression in a vicinity of a predetermined QRS peak.

6. Method according to claim 5, wherein the verifying is further based at least in part on one of the following: an RR interval, a PP interval, an RP interval, a correlation of a QRS complex morphology and/or on an ectopy probability, in the heart episode.

7. Method according to claim 5, wherein the verifying is performed by an artificial intelligence system and/or a machine learning system that has been trained with events that were verified via manual inspection of heart episodes.

8. Method according to claim 1 wherein, if the type comprises an asystole, high ventricular rate and/or a bradycardia, the verifying comprises eliminating, from the heart episode, RR intervals associated with a clipping of an amplitude, and preferably also RR intervals adjacent to at least one RR interval associated with a clipping of an amplitude.

9. Method according to claim 8, wherein the verifying further comprises:
detecting, in the heart episode, whether a pause associated with the asystole is present between subsequent QRS peaks, preferably corresponds to a (prolonged) RR interval that has not been eliminated.

10. Method according to claim 9, wherein the verifying further comprises:
if a pause is detected determining that the event is true positive otherwise determining that the event is false positive.

11. Method according to claim 1, wherein, if the type comprises a high ventricular rate and/or a bradycardia, the verifying comprises a calculation of a heart rate based on the heart episode.

12. Method according to claim 11, wherein the verifying is based at least in part on comparing the calculated heart rate with a predetermined threshold.

13. Apparatus for analyzing an event determined by an implantable device, wherein the apparatus is configured to perform a method of claim 1.

14. System for analyzing an event determined by an implantable device comprising:
an apparatus according to claim 13; and
the implantable device.

15. Computer program comprising instructions to perform a method of claim 1, when the instructions are executed.
